(19) United States
(12) Patent Application Publication
(10) Pub. No.: US 2020/0077960 A1
(43) Pub. Date: Mar. 12, 2020

(54) SYSTEMS AND METHODS FOR ALERTING A USER TO DETECTED EFFECTS OF A RECENT THERAPY ADJUSTMENT

(71) Applicant: MEDTRONIC MINIMED, INC., Northridge, CA (US)

(72) Inventors: Risako Morawiec, Santa Monica, CA (US); Key F. Payton, Burbank, CA (US); Ulrich H. Rankers, Livermore, CA (US); Steven J. Vargas, San Fernando, CA (US); Andrew Michael Bryan, San Diego, CA (US); David Legray, Northridge, CA (US)

(21) Appl. No.: 16/129,552

(22) Filed: Sep. 12, 2018

(51) Int. Cl. A61B 5/00; A61M 5/172; G16H 80/00; A61B 5/145; G16H 10/60

(52) U.S. Cl. CPC A61B 5/7275; A61M 5/1723; G16H 10/60; A61B 5/746; A61B 5/14532; G16H 80/00

(57) ABSTRACT

A method for confirming effectiveness of therapy provided by an insulin infusion system comprising a computing device communicatively coupled to an insulin delivery pump and a blood glucose sensor, is provided. In response to a corrective action for a detected abnormality in a blood glucose level of a patient, the method monitors a sequence of blood glucose levels of the patient, following the corrective action, by the computing device via the blood glucose sensor; detects an indication that the corrective action is effective to correct the abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and initiates presentation of an alert in response to the detected indication, by the computing device via a communicatively coupled display device.

### TECHNICAL FIELD

Subject matter disclosed herein relates to monitoring and/or controlling blood-glucose levels in patients.

### BACKGROUND

The pancreas of a normal healthy person produces and releases insulin into the bloodstream in response to elevated blood plasma glucose levels. Beta cells (β-cells), which reside in the pancreas, produce and secrete insulin into the bloodstream as it is needed. If j-cells become incapacitated or die, a condition known as Type 1 diabetes mellitus (or in some cases, if j-cells produce insufficient quantities of insulin, a condition known as Type 2 diabetes), then insulin may be provided to a body from another source to maintain life or health.

Traditionally, because insulin cannot be taken orally, insulin has been injected with a syringe. More recently, the use of infusion pump therapy has been increasing in a number of medical situations, including for delivering insulin to diabetic individuals. For example, external infusion pumps may be worn on a belt, in a pocket, or the like, and they can deliver insulin into a body via an infusion tube with a percutaneous needle or a cannula placed in subcutaneous tissue. An insulin infusion system may include an infusion pump that is automatically and/or semi-automatically controlled to infuse insulin into a patient. The infusion of insulin may be controlled to occur at times and in amounts that are based, for example, on blood glucose measurements obtained from an embedded glucose sensor in real time.

### BRIEF SUMMARY

Some embodiments of the present disclosure provide a method for confirming effectiveness of therapy provided by an insulin infusion system comprising a computing device communicatively coupled to an insulin delivery pump and a blood glucose sensor. In response to a corrective action for a detected abnormality in a blood glucose level of a patient, the method: (i) monitors a sequence of blood glucose levels of the patient, following the corrective action, by the computing device via the blood glucose sensor; (ii) detects an indication that the corrective action is effective to correct the abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and (iii) initiates presentation of an alert in response to the detected indication, by the computing device via a communicatively coupled display device.

Some embodiments of the present disclosure provide a medical device system comprising: an insulin delivery system; a glucose sensor system; and a computing device comprising a system memory element and at least one processor, the computing device communicatively coupled to the insulin delivery system and the glucose sensor system. The computing device is configured to: detect an abnormality in a blood glucose level of a patient, via the glucose sensor system; and in response to a corrective action for the abnormality, (i) monitor a sequence of blood glucose levels of the patient, following the corrective action, via the glucose sensor system; (ii) detect an indication that the corrective action is effective to correct the abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and (iii) in response to the detected indication, present an alert via the insulin delivery system.

Some embodiments of the present disclosure provide an insulin delivery system comprising: a glucose sensor system; and an insulin infusion device comprising an integrated computing device. The insulin infusion device is configured to: detect an abnormality in a blood glucose level of a patient, via the glucose sensor system; and in response to a corrective action for the abnormality, (i) monitor a sequence of blood glucose levels of the patient, following the corrective action, via the glucose sensor system; (ii) detect an indication that the corrective action is effective to correct the abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and (iii) in response to the detected turn, present an alert.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a block diagram of a glucose control system, in accordance with the disclosed embodiments;

FIGS. 2A-2C are diagrams of implementation embodiments of a computing device and insulin delivery system, in accordance with the disclosed embodiments;

FIG. 3 is a front view of blood glucose control hardware located on a body in accordance with the disclosed embodiments;

FIG. 4 is a plan view of a medical device component that includes a fluid infusion device, in accordance with the disclosed embodiments;

FIG. 5 is a functional block diagram of a computing device used as part of a glucose control system, in accordance with the disclosed embodiments;

FIG. 6 is a plot of a trajectory of a blood glucose trend including a significant direction change induced by a corrective action response to an abnormality, in accordance with the disclosed embodiments;

FIG. 7 is another plot of a trajectory of a blood glucose trend including a significant direction change induced by a corrective action response to an abnormality, in accordance with the disclosed embodiments;

FIG. 8 is a flow chart that illustrates an embodiment of a process for alerting a patient that a corrective action for a blood glucose abnormality has taken effect, in accordance with the disclosed embodiments;

FIG. 9 is a flow chart that illustrates an exemplary embodiment of a process for detecting an imprecise corrective action, in response to a detected blood glucose abnormality, in accordance with the disclosed embodiments; and

FIG. 10 is a flow chart that illustrates an embodiment of a process for detecting a significant direction change in a sequence of blood glucose levels, the significant direction change being a result of a corrective action response to an abnormality, in accordance with the disclosed embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

The subject matter presented herein relates to systems and methods for providing user notification of, or user alerts to, the effectiveness of a corrective action in response to an abnormality in detected blood glucose levels. More specifically, the subject matter relates to the detection of an indication of a change of direction of a blood glucose trend detected and reported by an insulin delivery pump and associated computing device. The change of direction of the blood glucose trend occurs when the corrective action is effective. The indication of the change of direction may be referred to as a “swerve”, “turn”, “curve”, or “inflection arc” in a graphical plot of blood glucose values over time, wherein the indication includes a plurality of sequential points where a first trend of the blood glucose values alternates direction to create a second trend progressing in an opposite direction of the first trend. Additionally contemplated herein is the presentation of an alert or notification to inform a user that a corrective action has produced the desired change to blood glucose values.

The non-limiting examples described herein relate to a medical device used to treat diabetes (more specifically, an insulin pump), although embodiments of the disclosed subject matter are not so limited. For the sake of brevity, conventional features and technologies related to infusion system operation, insulin pump and/or infusion set operation, blood glucose sensing and monitoring, sensor signal processing, and other functional aspects of the fluid-infusion system (and the individual operating components of the system) may not be described in detail here. Examples of fluid infusion devices, analyte sensors, and related components may be of the type described in, but not limited to, U.S. Pat. Nos. 6,659,980; 6,892,085; and 7,621,893 (which are incorporated by reference herein). Exemplary embodiments of an infusion-set component with integrated analyte sensor conductors are disclosed in United States patent publication number 2012/0238849 (which is incorporated by reference herein). An exemplary embodiment of a fluid-infusion device is disclosed in United States patent publication number 2015/0314068 (which is incorporated by reference herein).

Certain terminologies are used with regard to the various embodiments of the present disclosure. A sequence of blood glucose levels is a plurality of blood glucose values, acquired over a period of time, from a single patient or user of an insulin delivery pump. An abnormality in a sequence of blood glucose levels may include blood glucose levels below a predefined hypoglycemia threshold, blood glucose levels above a predefined hyperglycemia threshold, a decreasing blood glucose trend approaching or below a predefined hypoglycemia threshold, an increasing blood glucose trend approaching or above a predefined hyperglycemia threshold, or the like. The abnormality indicates that an intervention is required to maintain blood glucose stability at appropriate levels, and the intervention may include the administration of a bolus of insulin (to decrease hyperglycemic blood glucose levels) or the administration of a dose of carbohydrates (to increase hypoglycemic blood glucose levels). An imprecise corrective action to an abnormality in a sequence of blood glucose levels is a corrective action that does not adequately treat the blood glucose abnormality, such that additional corrective actions are necessary to bring the blood glucose levels of the patient within normal limits. Imprecise corrective actions may include under-correction or over-correction of abnormal blood glucose values.

Hypoglycemia is a deficiency of glucose in the bloodstream of the patient or user of the insulin delivery pump. Hypoglycemia may be generally associated with blood glucose values below 70 milligrams per deciliter (mg/dL), depending on certain patient-specific factors. Hyperglycemia is an excess of glucose in the bloodstream of the patient or user of the insulin delivery pump, and may be generally associated with blood glucose values above 180 mg/dL, depending on certain patient-specific factors. Normal, healthy blood glucose levels, over every 24-hour cycle, generally range between 70 and 160 mg/dL.

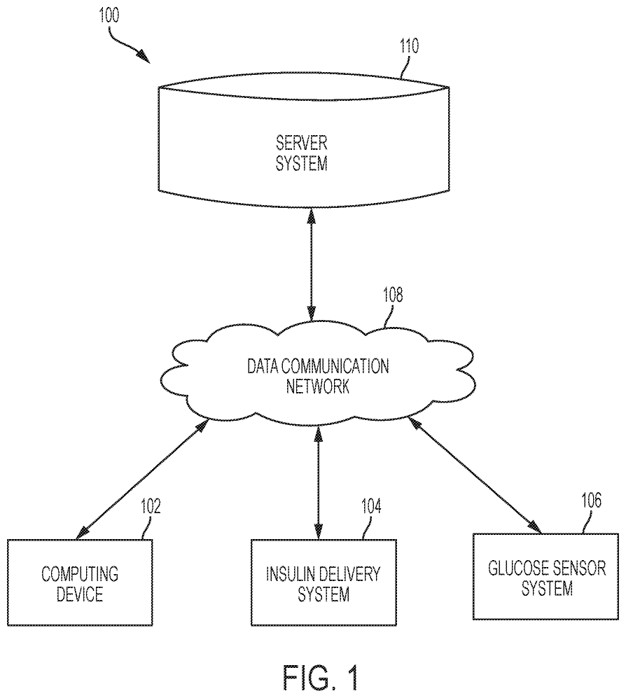

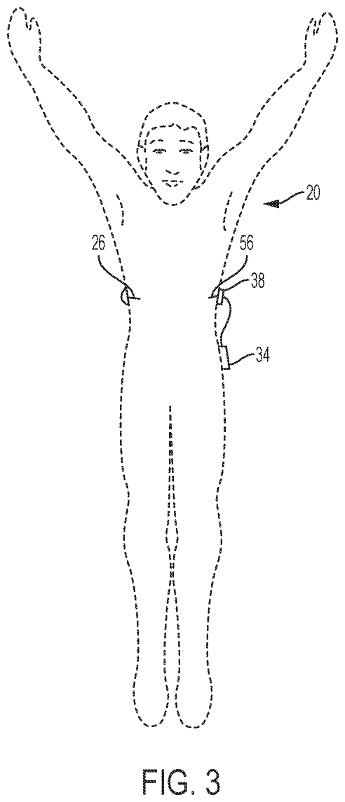

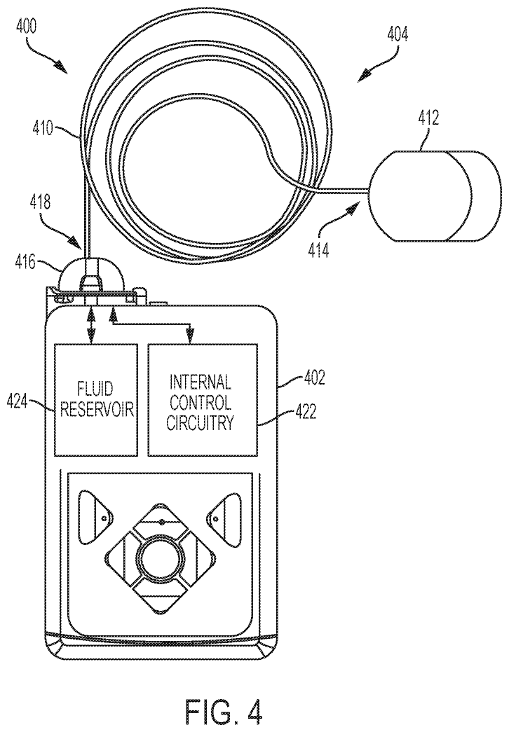

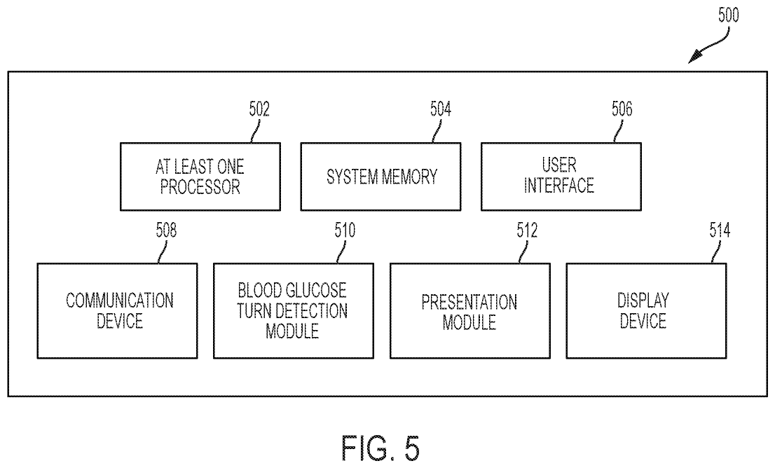

Turning now to the figures, FIG. 1 is a block diagram of a glucose control system 100, in accordance with the disclosed embodiments. Particular embodiments may include a glucose sensor system 106, a computing device 102, and an insulin delivery system 104, as shown in FIG. 1. Exemplary embodiments of the insulin delivery system 104 may be implemented using the system depicted in FIG. 4, exemplary embodiments of the computing device 102 may be implemented using the system depicted in FIG. 3, and exemplary embodiments of the glucose sensor system 106 may be implemented using the system depicted in FIG. 5. However, it should be appreciated that alternative embodiments of the insulin delivery system 104, the computing device 102, and/or the glucose sensor system 106 may be used within the context of the glucose control system 100 described in FIG. 1.

The glucose sensor system 106 generally includes a glucose sensor, sensor electrical components (to provide power to the glucose sensor and to generate glucose sensor signals), a sensor communication system (to transmit sensor signals to the computing device 102 and/or internal control circuitry of the insulin delivery system 104), and a sensor system housing for the electrical components and the sensor communication system. A glucose sensor may measure blood glucose directly from a blood stream, or indirectly via interstitial fluid using, e.g., a subcutaneous sensor. As used herein, “blood glucose”, “measured blood glucose”, “blood glucose concentration”, “measured blood glucose concentration”, and the like may refer to a glucose level, a blood glucose level, a blood glucose concentration, or the like, that has been obtained via any type of glucose sensor. It should be understood, however, that using a blood glucose sensor is only one particular technique for obtaining such observations or measurements, and that other techniques, such as measuring blood glucose via observations of other body fluids (e.g., observations of the presence of glucose in interstitial fluid using a subcutaneous sensor), may be used without deviating from claimed subject matter.

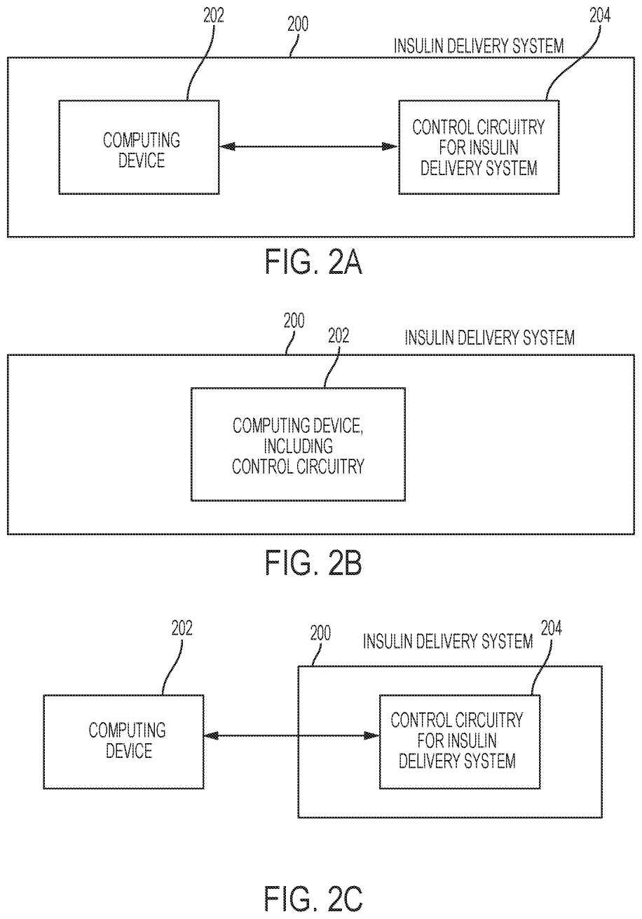

The computing device 102 may be implemented as an integrated component of the insulin delivery system 104 (e.g., an insulin delivery pump), or as an external component communicatively coupled to the insulin delivery system 104, as illustrated in FIGS. 2A-2C. FIG. 2A is an insulin delivery system 200 that includes a computing device 202 implemented as an integrated, internal component of the insulin delivery system 200. As shown, the computing device 202 is communicatively coupled to internal control circuitry 204 for the insulin delivery system 200, such that the computing device 202 transmits data to, and receives data from, the internal control circuitry 204 of the insulin delivery system 200. In this embodiment, the computing device 202 performs blood glucose monitoring and alert features following a corrective action (as described herein), and communicates with the internal control circuitry 204 to obtain data associated with standard operations of the insulin delivery system 200 (e.g., customary blood glucose control functions). FIG. 2B is a second embodiment of the insulin delivery system 200 that includes a computing device 202 implemented as an integrated, internal component of the insulin delivery system 200. However, as shown in FIG. 2B, the computing device 202 includes internal control circuitry for the insulin delivery system 200, such that the computing device 202 performs blood glucose monitoring and alert features following a corrective action (as described herein) in addition to controlling the standard operations of the insulin delivery system 200.

FIG. 2C is an embodiment of the insulin delivery system 200 that communicates with an external computing device 202 via wired or wireless communication connection. As shown, the insulin delivery system 200 includes internal control circuitry 204 to control the standard operations of the insulin delivery system 200, and the computing device 202 transmits data to, and receives data from, the internal control circuitry 204. In the embodiment shown in FIG. 2C, the computing device 202 is implemented as a component that is separate and distinct from the insulin delivery system 104, including but not limited to a computer system, a mobile computing device (e.g., a smartphone, a tablet computer, a wearable smart device, a laptop computer), a devoted patient monitor, or the like.

Exemplary embodiments of the computing device 202 shown in FIG. 2C are implemented as a mobile computing device that is configured to store, maintain, and execute mobile software applications (e.g., “apps”) applicable to blood glucose monitoring and alerting functionality. The external computing device 202, using an applicable blood glucose monitoring “app”, may receive and monitor blood glucose level data via a wired or wireless communication connection to the insulin delivery system 200 and blood glucose sensors (not shown). Thus, a patient may use the external computing device 202 and the activated blood glucose monitoring “app” to receive notifications and alerts related to the patient's blood glucose levels, blood glucose abnormalities, significant direction changes in a blood glucose trend resulting from a corrective action taken by the patient in response to an abnormality, or the like.

Returning to FIG. 1, as described with regard to FIGS. 2A-2B, the computing device 102 may be encased in an infusion device housing, and an infusion communication system may comprise an electrical trace or a wire that transmits commands from the computing device 102 to an infusion device. In alternative embodiments, the computing device 102 may be encased in a glucose sensor system 106 housing, and a glucose sensor communication system may comprise an electrical trace or a wire that carries glucose sensor signals from glucose sensor electrical components to computing device electrical components. As described with regard to FIG. 2C, the computing device 102 may include its own housing or may be included in a supplemental device. In yet other alternative embodiments, the computing device 102 may be co-located with an infusion device and a sensor system within a single housing. In further alternative embodiments, a glucose sensor, a computing device, and/or infusion communication systems may utilize a cable, a wire, a fiber optic line, radio frequency (RF), infrared (IR), or ultrasonic transmitters and receivers, combinations thereof, and/or the like instead of electrical traces, just to name a few examples.

The computing device 102 may include electrical components and software to generate commands for the insulin delivery system 104 based on a sensor signal received from the glucose sensor system 106. The computing device 102 may also include a communication system to receive sensor signals and to provide commands to the insulin delivery system 104. In exemplary implementations, the computing device 102 may include a user interface and/or operator interface (not shown) comprising a data input device and/or a data output device. Such a data output device may, for example, generate signals to initiate an alarm and/or include a display or printer for presenting a status of the computing device 102 and/or a patient's vital indicators. Such a data input device may comprise dials, buttons, pointing devices, manual switches, alphanumeric keys, a touch-sensitive display, combinations thereof, and/or the like for receiving user and/or operator inputs. Such a data input device may be used for scheduling and/or initiating insulin bolus injections for meals, for example. It should be understood, however, that these are merely examples of input and output devices that may be a part of an operator and/or user interface and that claimed subject matter is not limited in these respects.

The insulin delivery system 104 may include an infusion device and/or an infusion tube to infuse insulin into a patient's body. In particular embodiments, an infusion device (not explicitly identified in FIG. 1) may include infusion electrical components (to activate an infusion motor according to commands received from the computing device 102 and/or via a user interface of the infusion device), an infusion communication system (to receive commands from the computing device 102), and an infusion device housing (to hold the infusion device).

In the glucose control system 100 environment, blood-glucose measurements may be employed in an infusion system for regulating a rate of fluid infusion into a body. In particular embodiments, a glucose control system 100 may be adapted to regulate a rate of insulin into a patient's body based, at least in part, on a glucose concentration measurement taken from the patient's body (e.g., from the glucose sensor system 106 and/or metered blood glucose measurement). In certain implementations, such a glucose control system 100 may be designed to model a pancreatic beta cell (β-cell). Here, such a glucose control system 100 may enable a patient to control an infusion device (e.g., insulin delivery system 104) for releasing insulin into the patient's body for effective blood glucose management. In certain exemplary embodiments, glucose sensor system 106 generates a sensor signal representative of blood glucose levels in a patient's body, and the glucose sensor system 106 further provides a sensor signal to internal control circuitry of the insulin delivery system 104. The insulin delivery system 104 receives the sensor signal (from the glucose sensor system 106) and infuses insulin into the patient's body in response to the insulin-delivery system commands.

Embodiments of the computing device 102 are additionally configured to transmit data to, and receive data from, one or more remote servers (e.g., server system 110) or other storage medium. The server system 110 may include any number of application servers, and each server may be implemented using any suitable computer. In some embodiments, the server system 110 includes one or more dedicated computers. In some embodiments, the server system 110 includes one or more computers carrying out other functionality in addition to server operations. The server system 110 may store and provide any type of data relevant to the computing device 102, insulin delivery system 104, and/or the glucose sensor system 106. Such data may include, without limitation: blood glucose level data for a particular patient, blood glucose history data for one or more patients, glucose sensor data, insulin infusion data, recommended patient actions based on blood glucose levels and/or insulin infusion data, and other data compatible with the computing device 102.

The computing device 102 generally communicates with the server system 110 via a data communication network 108. The data communication network 108 may be any digital or other communications network capable of transmitting messages or data between devices, systems, or components. In certain embodiments, the data communication network 108 includes a packet switched network that facilitates packet-based data communication, addressing, and data routing. The packet switched network could be, for example, a wide area network, the Internet, or the like. In various embodiments, the data communication network 108 includes any number of public or private data connections, links or network connections supporting any number of communications protocols. The data communication network 108 may include the Internet, for example, or any other network based upon TCP/IP or other conventional protocols. In various embodiments, the data communication network 108 could also incorporate a wireless and/or wired telephone network, such as a cellular communications network for communicating with mobile phones, personal digital assistants, and/or the like. The data communication network 108 may also incorporate any sort of wireless or wired local and/or personal area networks, such as one or more IEEE 802.3, IEEE 802.16, and/or IEEE 802.11 networks, and/or networks that implement a short range (e.g., Bluetooth) protocol. For the sake of brevity, conventional techniques related to data transmission, signaling, network control, and other functional aspects of the systems (and the individual operating components of the systems) may not be described in detail herein.

During typical operation, the computing device 102 operates cooperatively with the insulin delivery system 104 and the glucose sensor system 106 to monitor blood glucose levels of a patient, and to identify abnormalities in the blood glucose levels. Abnormalities in blood glucose levels require intervention to bring the patient's blood glucose levels into a safe and healthy range. Interventions (i.e., corrective actions) may include administration of a bolus of insulin (to decrease blood glucose levels) or administration of a quantity of carbohydrates, glucose, or other medication (to increase blood glucose levels). The computing device 102 detects when a corrective action has been taken to correct an identified abnormality in the blood glucose levels. To do this, the computing device 102 determines whether a user has provided a user input indication that the user has self-administered carbohydrates or glucose, and whether the insulin delivery system 104 has delivered a bolus of insulin (whether automatically or in response to a user request).

After a corrective action has been taken, the computing device 102 monitors the patient's blood glucose levels to identify an indication of a change of direction of a blood glucose trend resulting from the corrective action. The indication of the change of direction may be referred to as a swerve, turn, curve, or inflection arc in a graphical plot of blood glucose values over time, wherein the indication includes a plurality of sequential points where a first trend of the blood glucose values alternates direction to create a second trend progressing in an opposite direction of the first trend. Thus, the indication of the change of direction is the location of the blood glucose trend at which decreasing blood glucose levels change direction to become increasing blood glucose levels, or at which increasing blood glucose levels change direction to become decreasing blood glucose levels. The computing device 102 then presents an alert, to notify the patient that the corrective action has taken effect and that the patient is no longer required to monitor blood glucose levels at the increased frequency necessitated by the administration of a corrective action.

FIG. 3 is a front view of glucose control system hardware located on a body 20, in accordance with the disclosed embodiments. Particular embodiments of the glucose control system hardware may include a sensor 26, an infusion device 34, and an infusion set 38, any or all of which may be worn on a body 20 of a user or patient, as shown in FIG. 2. It should be noted that the sensor 26 can be implemented with the glucose sensor system 106 depicted in FIG. 1. It should also be noted that the infusion device 34, the infusion set 38, and the cannula 56 can be implemented with the insulin delivery system 104 depicted in FIG. 1. In this regard, the glucose control system hardware shows certain elements and components of the insulin delivery system 104 and the glucose sensor system 106 in more detail.

With reference to FIG. 1, a computing device 102 and/or internal circuitry of the insulin delivery system 104 may process received sensor values and generate commands for the infusion device (e.g., insulin delivery system 104). The insulin delivery system 104 may respond to commands and actuate a plunger that forces insulin out of a reservoir that is located inside an infusion device. In particular example embodiments, a connector tip of a reservoir may extend through an infusion device housing, and a first end of an infusion tube may be attached to connector tip. A second end of the infusion tube may connect to infusion set 38 (e.g., of FIG. 2). The infusion set 38 includes a tube, a connector that physically and fluidly couples to the infusion device 34, which is affixed to the skin, and the cannula 56 (e.g., a soft tube or needle) that penetrates the skin. Insulin may be forced through infusion tube into infusion set 38 and into body 20. Infusion set 38 may be adhesively attached to a user's skin. As part of infusion set 38, a cannula 56 may extend through skin and terminate in subcutaneous tissue to complete fluid communication between a reservoir and subcutaneous tissue of a user's body 20.

As shown, the glucose control system hardware may be used, for example, in controlling a patient's glucose level, identifying current blood glucose levels and providing instructions for appropriate corrective actions applicable to the current blood glucose levels, and using a communicatively coupled computing device (see reference 102, FIG. 1; reference 300, FIG. 3) to detect a turn, swerve, inflection arc, or other indication of a change of direction in a sequence of blood glucose levels that has occurred based on a completed corrective action. It should be understood, however, that these are merely examples of particular systems that may be used for providing alerts for a blood glucose control system and that claimed subject matter is not limited in this respect.

FIG. 4 is a plan view of a fluid infusion system 400 that includes a fluid infusion device 402, in accordance with the disclosed embodiments. It should be noted that fluid infusion system 400 can be utilized in the context of the system shown in FIG. 1. In this regard, fluid infusion system 400 shows certain elements and components of the insulin delivery system 104 in more detail.

The fluid infusion system 400 is used for infusing medication fluid into the body of a user, to treat a medical condition of a patient. As shown, the fluid infusion system 400 includes two primary elements: the fluid infusion device 402 (e.g., an insulin pump) and an infusion set component 404, which can be coupled to the fluid infusion device 402 as depicted in FIG. 4. This particular embodiment of the infusion set component 404 includes, without limitation: an infusion tube 410; an infusion unit 412 coupled to one end 414 of the infusion tube 410; and a connector assembly 416 coupled to the other end 418 of the infusion tube 410. The fluid infusion device 402 is designed to be carried or worn by the patient, and the infusion set component 404 terminates at the infusion unit 412 such that the fluid infusion device 402 can deliver medication fluid to the body of the patient in a controlled and regulated manner via the infusion tube 410. The fluid infusion device 402 may leverage a number of conventional features, components, elements, and characteristics of existing fluid infusion devices. For example, the fluid infusion device 402 may incorporate some of the features, components, elements, and/or characteristics described in U.S. Pat. No. 7,621,893 and United States patent publication number 2015/0314068, the relevant content of which is incorporated by reference herein.

When realized as an insulin infusion pump, the fluid infusion device 402 controls and manages the delivery of insulin to manage blood glucose levels of the patient. The fluid infusion device 402 accommodates the fluid reservoir 424 that contains the medication fluid to be delivered to the user. The infusion tube 410 represents the fluid flow path that couples the fluid reservoir 424 to the infusion unit 412. When installed as depicted in FIG. 4, the infusion tube 410 extends from the fluid infusion device 402 to the infusion unit 412, which in turn provides a fluid pathway to the body of the patient. For the illustrated embodiment, the connector assembly 416 is realized as a removable reservoir cap (or fitting) that is suitably sized and configured to accommodate replacement of fluid reservoirs 424 (which are typically disposable) as needed. In this regard, the reservoir cap is designed to accommodate the fluid path from the fluid reservoir 424 to the infusion tube 410. Accordingly, the fluid reservoir 424 is fluidly coupled to the infusion tube 410, by way of the connector assembly 416.

The medical device component (e.g., fluid infusion device 402) operates to regulate the delivery of medication fluid to the patient. The fluid infusion device 402 generally includes internal control circuitry 422 that controls a mechanism (not shown) to actuate a fluid reservoir 424 housed in the body of the fluid infusion device 402. The fluid infusion system 400 operates cooperatively with an internal or an external computing device (e.g., reference 102, FIG. 1; reference 500, FIG. 5) to detect, and provide alerts regarding, a turn, swerve, inflection arc, or other indication of a change of direction in a sequence of blood glucose levels, wherein the indication of the change occurs as a result of a corrective action performed by a user in combination with a user input indication that the corrective action has been taken.

As described previously with regard to FIGS. 2A-2C, the internal control circuitry 422 of the fluid infusion device 402 may be implemented as control circuitry configured to operate cooperatively with an internal computing device (see FIG. 2A), wherein the internal control circuitry 422 functions to control standard operations of the fluid infusion device 402 and the computing device functions to performs blood glucose monitoring and alert features following a corrective action (as described herein). Alternatively, the internal control circuitry 422 of the fluid infusion device 402 may be implemented as an internal computing device that includes control circuitry (see FIG. 2B), wherein the computing device functions to control standard operations of the fluid infusion device 402 in addition to blood glucose monitoring and alert features following a corrective action. Thus, the computing device may be integrated into the fluid infusion system 400, such that the fluid infusion system 400 and the computing device are housed in a single casing.

In other embodiments, the internal control circuitry 422 of the fluid infusion device 402 may be implemented as control circuitry configured to operate cooperatively with an external computing device (see FIG. 2C). Like the embodiment illustrated in FIG. 2A, in this embodiment the internal control circuitry 422 functions to control standard operations of the fluid infusion device 402 and the computing device functions to performs blood glucose monitoring and alert features following a corrective action. Thus, the computing device is communicatively coupled to the fluid infusion system 400 via wired or wireless communication connection, wherein the computing device and the medical device component (e.g., fluid infusion system 400) are separate and distinct devices. In this example, the computing device may be implemented as a standalone computing device, such as a laptop computer, a tablet computer, a smartphone, a cloud computing system, a remotely located server system, or the like.

The fluid infusion system 400 can use any of the above-described implementations of the internal control circuitry 422 and an associated computing device, (i) to provide alerts associated with corrective action effectiveness, as described herein, and (ii) to perform standard operations of the fluid infusion system 400 (e.g., to regulate the delivery of medication fluid to the patient). To provide alerts associated with corrective action effectiveness, the computing device operates cooperatively with the internal control circuitry 422 of the fluid infusion system 400 to monitor blood glucose levels of a patient, to identify abnormalities in the blood glucose levels, to receive user input indicating that a corrective action has been taken, and to identify an indication of a change of direction (i.e., a swerve, turn, curve, inflection arc, or the like) in the sequence of blood glucose values resulting from the corrective action.

FIG. 5 is a functional block diagram of a computing device 500 or processor-based component that can be used as part of a glucose control system, in accordance with the disclosed embodiments. It should be noted that the computing device 500 can be implemented as the computing device 102 depicted in FIG. 1. Thus, in some embodiments, the computing device 500 may be implemented as an integrated and internal processor-based component of an insulin delivery pump. In alternative embodiments, the computing device 500 can be implemented as a distinct hardware device that operates cooperatively with an insulin delivery pump, such as a monitor device, a controller device, a mobile computing device, or the like.

The computing device 500 generally includes, without limitation: at least one processor 502; system memory 504; a user interface 506; a communication device 508; a blood glucose turn detection module 510; a presentation module 512; and a display device 514. These elements and features of the computing device 500 may be operatively associated with one another, coupled to one another, or otherwise configured to cooperate with one another as needed to support the desired functionality—in particular, dynamically providing an alert feature for an insulin delivery system associated with a patient, which provides alerts or notifications to the patient when a corrective action has achieved a desired effect on the patient's blood glucose trend, as described herein. For ease of illustration and clarity, the various physical, electrical, and logical couplings and interconnections for these elements and features are not depicted in FIG. 5. Moreover, it should be appreciated that embodiments of the computing device 500 will include other elements, modules, and features that cooperate to support the desired functionality. For simplicity, FIG. 5 only depicts certain elements that relate to the corrective action effectiveness detection and alerting techniques described in more detail below.

The at least one processor 502 may be implemented or performed with one or more general purpose processors, a content addressable memory, a digital signal processor, an application specific integrated circuit, a field programmable gate array, any suitable programmable logic device, discrete gate or transistor logic, discrete hardware components, or any combination designed to perform the functions described here. In particular, the at least one processor 302 may be realized as one or more microprocessors, controllers, microcontrollers, or state machines. Moreover, the at least one processor 302 may be implemented as a combination of computing devices, e.g., a combination of digital signal processors and microprocessors, a plurality of microprocessors, one or more microprocessors in conjunction with a digital signal processor core, or any other such configuration.

The at least one processor 502 is communicatively coupled to, and operates cooperatively with, the system memory 504 element. The system memory 504 is configured to store any obtained or generated data associated with blood glucose levels of a patient, including current and historical blood glucose levels, blood glucose level threshold data (e.g., hypoglycemic threshold, hyperglycemic threshold) for a patient, blood glucose trend direction change data (e.g., indication data, sequential points of the indication, slope values between sequential points, direction change slope threshold data, timing data, corrective action data, sampling rate data), and historical alert data. The system memory 504 may be realized using any number of devices, components, or modules, as appropriate to the embodiment. Moreover, the computing device 500 could include system memory 504 integrated therein and/or a system memory 504 operatively coupled thereto, as appropriate to the particular embodiment. In practice, the system memory 504 could be realized as RAM memory, flash memory, EPROM memory, EEPROM memory, registers, a hard disk, a removable disk, or any other form of storage medium known in the art. In certain embodiments, the system memory 504 includes a hard disk, which may also be used to support functions of the computing device 500. The system memory 504 can be coupled to the at least one processor 502 such that the at least one processor 502 can read information from, and write information to, the system memory 504. In the alternative, the system memory 504 may be integral to the at least one processor 502. As an example, the at least one processor 502 and the system memory 504 may reside in a suitably designed application-specific integrated circuit (ASIC).

The user interface 506 may include or cooperate with various features to allow a user to interact with the computing device 500 and/or an insulin pump device that includes the computing device 500 as an integrated component. Accordingly, the user interface 506 may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the computing device 500 and/or insulin pump device. For example, the user interface 506 could be manipulated by an operator to provide user-initiated corrective action data (e.g., an indication that a user has ingested a dose of glucose or a quantity of carbohydrates), or a user input acknowledgement of an alert or notification, as described herein.

In certain embodiments, the user interface 506 may include or cooperate with various features to allow a user to interact with the computing device 500 and/or insulin pump device via graphical elements rendered on a display element (e.g., the display device 514). Accordingly, the user interface 506 may initiate the creation, maintenance, and presentation of a graphical user interface (GUI). In certain embodiments, the display device 514 implements touch-sensitive technology for purposes of interacting with the GUI. Thus, a user can manipulate the GUI by moving a cursor symbol rendered on the display device 514, or by physically interacting with the display device 514 itself for recognition and interpretation, via the user interface 506.

The communication device 508 is suitably configured to communicate data between the computing device 500 and one or more remote servers and one or more glucose sensor systems used to detect blood glucose levels of a patient. The communication device 508 may transmit and receive communications over a wireless local area network (WLAN), the Internet, a satellite uplink/downlink, a cellular network, a broadband network, a wide area network, or the like. As described in more detail below, the communication device 508 receives current blood glucose level data from one or more glucose sensors associated with a patient. Additionally, the communication device 508 receives data from a remotely located server system, local system memory 504, or other remotely located storage medium, including, but not limited to: blood glucose level history data, blood glucose level threshold data (e.g., hypoglycemia threshold data and hyperglycemia threshold data associated with a particular patient), blood glucose level trend data, blood glucose level alert data (e.g., alert history, alert thresholds), and other data compatible with the computing device 500. The communication device 508 also transmits data to at least one storage medium (e.g., server system, local system memory 504), including but not limited to: current blood glucose level data, breached threshold data (e.g., a breached hypoglycemia threshold, a breached hyperglycemia threshold), timing data associated with blood glucose levels, notification or alert data, or the like.

The blood glucose turn detection module 510 is configured to detect an indication of a change of direction in a sequence of blood glucose levels requiring correction, wherein the indication shows that a previous corrective action to correct a blood glucose abnormality has been successful. The indication may be referred to as a turn, swerve, curve, inflection arc, or the like. The indication generally includes a significant and sharp swerve, curve, or turn in the sequence of blood glucose levels, wherein a first trend in the sequence of blood glucose levels alternates direction at the indication (from a downward-progressing trend to an upward-progressing trend, or from an upward-progressing trend to a downward-progressing trend) to create a second trend in blood glucose levels, and wherein the second trend progresses in an opposite direction of the first trend. Thus, the indication is the location of the blood glucose trend at which decreasing blood glucose levels change direction to become increasing blood glucose levels, or at which increasing blood glucose levels change direction to become decreasing blood glucose levels.

The blood glucose turn detection module 510 is configured to monitor blood glucose values for an indication of a change of direction of a blood glucose trend in response to received user input data (via the user interface 506) communicating that the user has taken a corrective action for a blood glucose abnormality. The blood glucose turn detection module 510 of the computing device 500 monitors a patient's blood glucose levels via a blood glucose sensor (reference 106, FIG. 1), and determines whether an abnormality is present in the sequence of blood glucose levels. An abnormality in a sequence of blood glucose levels may include blood glucose levels below a predefined hypoglycemia threshold, blood glucose levels above a predefined hyperglycemia threshold, a decreasing blood glucose trend approaching or below a predefined hypoglycemia threshold, an increasing blood glucose trend approaching or above a predefined hyperglycemia threshold, or the like. The abnormality indicates that a corrective action is required to maintain blood glucose stability at appropriate levels, and the corrective action may include the administration of a bolus of insulin (to decrease hyperglycemic blood glucose levels) or the administration of a dose of carbohydrates (to increase hypoglycemic blood glucose levels). After a corrective action has been taken by a user, and the user has provided user input data (via the user interface 506) communicating that the user has taken a corrective action for a blood glucose abnormality, the blood glucose turn detection module 510 increases a sampling rate of the blood glucose sensor to monitor the patient's blood glucose levels on a more frequent basis.

The blood glucose turn detection module 510 monitors for the indication of a change of direction of the blood glucose trend by increasing the sampling rate of the blood glucose sensor and, using the increased sampling rate, detecting a plurality of sequential points of the blood glucose trend exceeding a predetermined slope threshold value. The predetermined slope threshold value is a minimum value for blood glucose levels indicating a change in direction for the blood glucose trend resulting from a user-performed corrective action. The blood glucose turn detection module 510 determines whether detected blood glucose values for a plurality of sequential points exceed the predetermined slope threshold value, and if so, determines that the plurality of sequential points are the indication of the change of direction for the blood glucose trend.

In practice, the blood glucose turn detection module 510 may be implemented with (or cooperate with) the at least one processor 502 to perform at least some of the functions and operations described in more detail herein. In this regard, the blood glucose turn detection module 510 may be realized as suitably written processing logic, application program code, or the like.

The presentation module 512 is configured to present (via the display device 514) graphical elements and text associated with notifications and alerts for the glucose control system (see reference 100, FIG. 1). The presentation module 512 identifies appropriate graphical elements for presentation via the display device 514, based on parameters used by, and data obtained by, the blood glucose turn detection module 510. The presentation module 512 is configured to identify a current sampling rate, a current blood glucose level, a detected indication of a change of direction in a current sequence of blood glucose levels, and to present applicable graphical elements and text based on the current sampling rate, current blood glucose level, and the detected indication of the change of direction, via the display device 514.

The presentation module 512 is further configured to provide alerts and notifications applicable to a detected indication of a change of direction in the sequence of blood glucose levels, timing data associated with a detected indication of a change of direction in the sequence of blood glucose levels, an indication that a corrective action should be taken by a user, confirmation of a user-input indication that a corrective action has been completed by a user, and the like. Notifications and alerts provided by the presentation module 512 may include graphical elements and text presented via the display device 514, as described previously. However, it should be appreciated that the presentation module 512 also initiates other types of alerts presented by an insulin pump device associated with the computing device 500 and/or presented via a communicatively coupled personal computing device (e.g., a laptop computer, a tablet computer, a smartphone). Such notifications and alerts may include, without limitation: audio alerts (e.g., sound effects, articulated speech alerts, alarms), visual alerts (e.g., text effects, flashing or otherwise activated lights, color changes), and/or any other type of alert generated to attract the attention of the user.

The display device 514 is configured to display various icons, text, and/or graphical elements associated with blood glucose levels of a patient. In an exemplary embodiment, the display device 514 and the user interface 506 are communicatively coupled to the at least one processor 502. The at least one processor 502, the user interface 506, and the display device 514 are cooperatively configured to display, render, or otherwise convey one or more graphical representations or images associated with blood glucose levels, an indication of a change of direction in a blood glucose trend, and/or notifications or alerts associated with blood glucose levels on the display device 514, as described in greater detail below. In an exemplary embodiment, the display device 514 is realized as an electronic display configured to graphically display blood glucose trend data, including alerts and notifications associated with a change in direction of, as described herein.

In some embodiments, the computing device 500 is an integrated computer system that is included as part of an insulin pump device, wherein the display device 514 is located inside a housing of the insulin pump device, and is thus implemented as an insulin pump display. In other embodiments, the display device 514 is implemented as a display screen of a standalone, personal computing device (e.g., laptop computer, tablet computer, smartphone) or other medical device (e.g., an external monitor device), wherein the display device 514 is communicatively coupled to the computing device 500 via a wired and/or wireless communication connection. It will be appreciated that although the display device 514 may be implemented using a single display, certain embodiments may use additional displays (i.e., a plurality of displays) to accomplish the functionality of the display device 514 described herein.

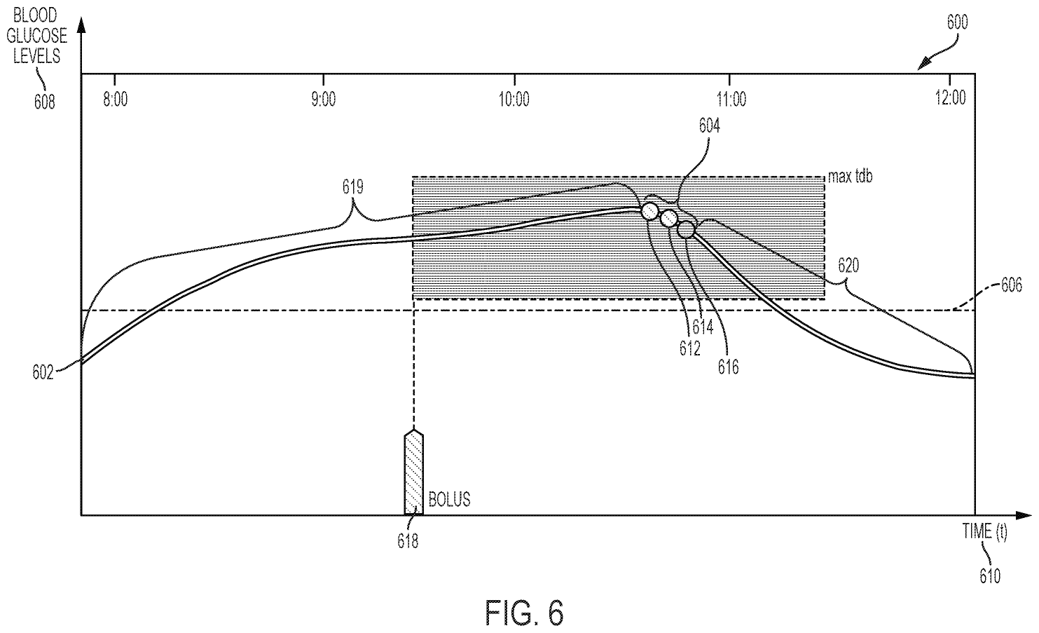

FIG. 6 is a plot 600 of blood glucose levels 608 over time 610. FIG. 6 shows a trajectory of blood glucose levels 608 (i.e., a sequence of blood glucose levels, a blood glucose trend 602) including an indication 604 of a change of direction induced by a corrective action response to an abnormality, in accordance with the disclosed embodiments. The plot 600 shows the glucose trend 602 as a plurality of blood glucose levels 608, for a particular user, over time 610. The blood glucose levels 608 associated with the glucose trend 602 are obtained by a blood glucose sensor (e.g., reference 106, FIG. 1) of an insulin delivery pump (e.g., reference 104, FIG. 1). As described previously with regard to FIGS. 1-5, the blood glucose sensor and the insulin delivery pump are communicatively coupled to a computing device (e.g., reference 102, FIG. 1; reference 500, FIG. 5) that stores, maintains, and executes processing instructions to obtain and analyze the plurality of blood glucose levels 608, and to initiate presentation of alerts and notifications associated with the plurality of blood glucose levels 608. Embodiments of the computing device may be implemented as an integrated part of an insulin pump device, or as a standalone personal computing device communicatively coupled to the insulin pump device via wired and/or wireless connection (e.g., a laptop computer, a tablet computer, a smartphone, a remotely located “cloud” computer system or server system, a computer system located in a physician's office or other healthcare facility).

As shown, the glucose trend 602 (i.e., the sequence of blood glucose levels) is increasing, from below a hyperglycemia threshold 606 to above the hyperglycemia threshold 606. In the example shown, the glucose trend 602 continues to increase above the hyperglycemia threshold 606. However, it should be appreciated that a glucose trend 602 may not continue increasing, and blood glucose values may simply be detected above the hyperglycemia threshold 606, whether in an increasing, decreasing, or a constant (or relatively constant) trend of blood glucose values.

The plot 600 illustrates the timing of an administration of a bolus of insulin 618, wherein the bolus of insulin 618 is administered in response to either: (1) an increasing blood glucose trend 602 of blood glucose values above a predefined hyperglycemia threshold 606; or (2) a blood glucose trend 602 that exists above the predefined hyperglycemia threshold 606, but that is not necessarily an increasing trend. The plot 600 depicts one particular instance where, after a bolus of insulin 618 is administered, an insulin delivery pump and associated computing device continue to monitor the sequence of blood glucose levels (i.e., the blood glucose trend 602) to identify an indication 604 of a change of direction in the blood glucose trend 602. The indication 604 of a change of direction is a turn, swerve, curve, or inflection arc in the sequence of blood glucose levels (i.e., the blood glucose trend 602), wherein a first trend 619 in the sequence of blood glucose levels alternates direction at the indication 604 to create a second trend 620 in blood glucose levels. In the embodiment shown, the first trend 619 (which is increasing) progresses in an opposite direction of the second trend 620 (which is decreasing).

The indication 604 of the change of direction of the blood glucose trend 602 is identified by the computing device based on slope values between points (i.e., between discrete blood glucose values) of the blood glucose trend 602. The slope between two points of the glucose trend describes the steepness of the glucose trend between the two points, and is calculated using well-known and commonly used techniques. For example, when a first sequential point and a second sequential point of the blood glucose trend 602 are detected consecutively, via the blood glucose sensor, the slope between the first sequential point and the second sequential point may be calculated as a ratio of vertical change to horizontal change between the two points. The indication 604 of the change of direction of the blood glucose trend 602 is identified by comparing slope values between sequential points of the blood glucose trend 602 to a predetermined slope threshold and detecting that one or more of the slope values exceed the predetermined slope threshold. The predetermined slope threshold is generally defined as X mg/dL/min, wherein X is preconfigured and stored for use in detecting effectiveness of corrective actions performed by a user. In various embodiments of the present disclosure, the computing device may compare a slope value or an absolute value of a slope value a predetermined slope threshold to identify the indication 604.

As described herein, the slope values used to identify the indication 604 of the change of direction of the blood glucose trend 602 are based on discrete blood glucose values obtained by the blood glucose sensor. The computing device initiates sampling of the patient's blood glucose levels via the blood glucose sensor, using a default or standard sampling rate. The sampling rate is the frequency at which a discrete blood glucose level of a patient is obtained, via the blood glucose sensor. Generally, the default sampling rate is chosen to preserve battery life of the insulin pump and blood glucose sensor. In one example, a standard sampling rate of a blood glucose sensor is five (5) minutes.

In certain embodiments, the insulin delivery pump and associated computing device increase the sampling rate to create an increased sampling rate, in response to receiving a user input data communication that the user has self-administered a corrective action for a blood glucose abnormality, wherein the increased sampling rate is used to identify the indication 604 of the change of direction of the blood glucose trend 602. In practice, the identification of the indication 604 requires more samples per unit of time than the quantity of samples obtainable using a 5-minute time interval for sampling. Thus, the computing device increases the sampling rate, such that the blood glucose sensor uses a shorter interval of time. Exemplary embodiments of the blood glucose control system (e.g., reference 100, FIG. 1) use a one (1) minute time interval. However, it should be appreciated that the increased sampling rate may be implemented using any time interval that is shorter than the standard sampling rate of the blood glucose sensor.

In the exemplary embodiment shown, the indication 604 of the change in direction is detected via at least three samples: a first sample 612, a second sample 614, and a third sample 616. However, it should be appreciated that other embodiments may use fewer samples or a greater number of samples, depending on the application. As shown, the second sample 614 detects a lower blood glucose level than the first sample 612, and the third sample 616 detects a lower blood glucose level than the second sample 614. Thus, the first sample 612 detects a highest blood glucose level of the increasing blood glucose trend 602; the second sample 614 detects a blood glucose level that has decreased since the first sample 612; and the third sample 616 detects a blood glucose level that has decreased from the second sample 614 and thus has further decreased from the first sample 612. A first slope value describes the steepness of the blood glucose trend 602 between the first sample 612 and the second sample 614, and a second slope value describes the steepness of the blood glucose trend 602 between the second sample 614 and the third sample 616. In the exemplary embodiment shown, the first slope value and the second slope value exceed a predetermined slope threshold, and thus the plurality of sequential points (e.g., the first sample 612, the second sample 614, and the third sample 616) collectively comprise the indication 604 of the direction change of the blood glucose trend 602. The indication 604 (which includes blood glucose levels detected by the first sample 612, the second sample 614, and the third sample 616) is a significant change in direction of the blood glucose trend 602 and therefore serves as an indication that the administered bolus of insulin 618 has produced the desired effect. In this example, the desired effect is to decrease the blood sugar level of the patient. In other words, the indication 604 illustrates that the administered bolus of insulin 618 has changed the direction of the blood glucose trend from an increasing trend that continues to increase above the hyperglycemia threshold 606.

In response to the detected indication 604 of the change of direction for the blood glucose trend 602, the insulin delivery pump and associated computing device provide an alert, such that the patient is informed that the administered bolus of insulin 618 has taken effect. Generally, a bolus of insulin 618 is either administered automatically (in response to a detected abnormality in blood glucose trend 602, such as an increasing blood glucose trend 602 that continues to increase beyond a hyperglycemia threshold 606) or in response to a user input command to the insulin delivery pump. Here, the detected indication 604 is reported to the patient (via alert or notification) to provide awareness to the patient that the bolus of insulin 618 has been effective in changing the direction of the blood glucose trend 602.

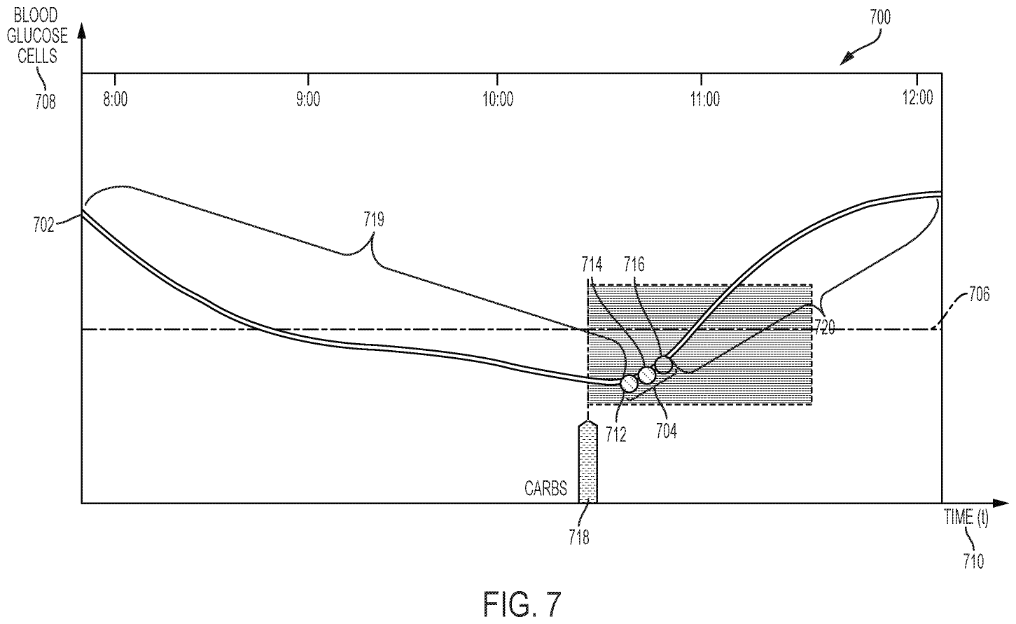

FIG. 7 is another plot of a trajectory of blood glucose levels 708 (i.e., a sequence of blood glucose levels, a blood glucose trend 702), including an indication 704 of a change of direction in the blood glucose trend 702 induced by a corrective action response to an abnormality, in accordance with the disclosed embodiments. The plot 700 shows the glucose trend 702 as a plurality of blood glucose levels 708, for a particular user, over time 710. As described previously with regard to FIGS. 1-6, the blood glucose levels 708 associated with the glucose trend 702 are obtained by a blood glucose sensor (e.g., reference 106, FIG. 1) of an insulin delivery pump (e.g., reference 104, FIG. 1), which are communicatively coupled to a computing device (e.g., reference 102, FIG. 1; reference 300, FIG. 3) that stores, maintains, and executes processing instructions to obtain and analyze the plurality of blood glucose levels 708, and to initiate presentation of alerts and notifications associated with the plurality of blood glucose levels 708.

As shown, the glucose trend 702 (i.e., the sequence of blood glucose levels) is decreasing, from above a hypoglycemia threshold 706 to below the hypoglycemia threshold 706. In the embodiment shown, the glucose trend 702 continues to decrease below the hypoglycemia threshold 706. However, it should be appreciated that a glucose trend 702 may not continue decreasing, and blood glucose values may simply be detected below the hypoglycemia threshold 706, whether in an increasing, decreasing, or a constant (or relatively constant) trend of blood glucose values.

The plot 700 illustrates the administration of a dose of carbohydrates 718, wherein the dose of carbohydrates 718 is administered in response to either: (1) a decreasing blood glucose trend 702 of blood glucose values below a predefined hypoglycemia threshold 706; or (2) a blood glucose trend 702 that exists below the predefined hypoglycemia threshold 706, but that is not necessarily a decreasing trend. The plot 700 depicts one particular instance where, after a dose of carbohydrates 718 is administered, an insulin delivery pump and associated computing device continues to monitor the sequence of blood glucose levels (i.e., the blood glucose trend 702) to identify an indication 704 of a change of direction in the blood glucose trend 702. The indication 704 is a turn, curve, swerve, or inflection arc in the sequence of blood glucose levels (i.e., the blood glucose trend 702), wherein a first trend 719 in the sequence of blood glucose levels alternates direction at the indication 704 to create a second trend 720 in blood glucose levels. In the embodiment shown, the first trend 719 (which is decreasing) progresses in an opposite direction of the second trend 720 (which is increasing).

As explained previously, in certain embodiments, the insulin delivery pump and associated computing device increase a sampling rate of the sequence of blood glucose levels (i.e., the blood glucose trend 702), to create an increased sampling rate of sensor glucose values, wherein the increased sampling rate is used to identify the indication 704 of the change of direction in the blood glucose trend 602. In the exemplary embodiment shown, the indication 704 is detected via at least three samples: a first sample 712, a second sample 714, and a third sample 716. However, it should be appreciated that other embodiments may use fewer samples or a greater number of samples, depending on the application. As shown, the second sample 714 detects a higher blood glucose level than the first sample 712, and the third sample 716 detects a higher blood glucose level than the second sample 714. Thus, the first sample 712 detects a lowest blood glucose level of the decreasing blood glucose trend 702; the second sample 714 detects a blood glucose level that has increased since the first sample 712; and the third sample 716 detects a blood glucose level that has increased from the second sample 714 and thus has further increased from the first sample 712. A first slope value describes the steepness of the blood glucose trend 702 between the first sample 712 and the second sample 714, and a second slope value describes the steepness of the blood glucose trend 702 between the second sample 714 and the third sample 716. In the exemplary embodiment shown, the first slope value and the second slope value exceed a predetermined slope threshold, and thus the plurality of sequential points (e.g., the first sample 712, the second sample 714, and the third sample 716) collectively comprise the indication 704 of the direction change of the blood glucose trend 702. The indication 704 (which includes blood glucose levels detected by the first sample 712, the second sample 714, and the third sample 716) is a change of direction of the blood glucose trend 702 and therefore serves as an indication that the administered dose of carbohydrates 718 has produced the desired effect. In this example, the desired effect is to increase the blood sugar level of the patient. In other words, the indication 704 illustrates that the administered dose of carbohydrates 718 has changed the direction of the blood glucose trend from a decreasing trend that continues to decrease below the hypoglycemia threshold 706.

In response to the detected indication of the change of direction, the insulin delivery pump and associated computing device provide an alert, such that the patient is informed that the administered dose of carbohydrates 718 has taken effect. Generally, a dose of carbohydrates 718 is administered by a user in response to a detected (and communicated by the insulin delivery pump) abnormality in blood glucose trend 702, such as a decreasing blood glucose trend 702 that continues to decrease beyond a hypoglycemia threshold 706. Here, the detected indication 704 is reported to the patient (via alert or notification) to provide awareness to the patient that the dose of carbohydrates 718 has been effective in changing the direction of the blood glucose trend 702. Alerts or notifications provided or presented by the computing device may include auditory, visual, and/or vibratory alerts that provide a clear communication to the patient that the corrective action administered by the patient was effective. In some embodiments, the computing device may display a graphical plot of the blood glucose trend 702 in conjunction with a visual alert wherein the portion of graph including the indication 704 of the change of direction for the blood glucose trend 702 is highlighted or otherwise presented using visual characteristics that are distinguishable from the rest of the graph. In some embodiments, the computing device generates and transmits a text message or email to a designated set of contact information, which may be configurable for the patient.

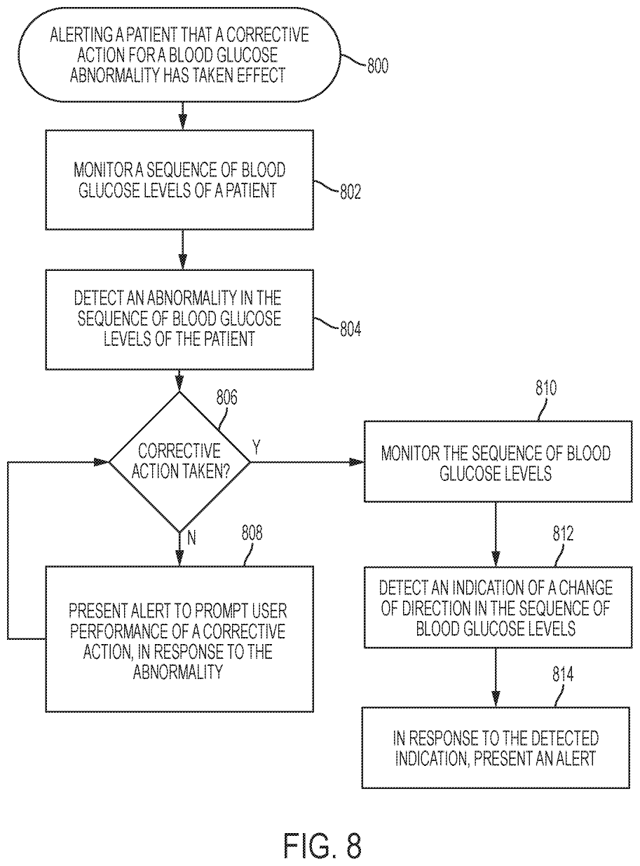

FIG. 8 is a flow chart that illustrates an embodiment of a process 800 for alerting a patient that a corrective action for a blood glucose abnormality has taken effect, in accordance with the disclosed embodiments. For ease of description and clarity, the process 800 begins by monitoring a sequence of blood glucose levels of a patient (step 802). As described previously, with regard to FIGS. 1-7, exemplary embodiments of the process 800 use an insulin delivery pump and associated computing device, which are communicatively coupled to a subcutaneous sensor and are monitoring blood glucose levels of the user of the insulin delivery pump via the subcutaneous sensor. In certain embodiments, the process 800 obtains blood glucose levels for the user, via the subcutaneous sensors, at timed intervals. In some embodiments, however, the process 800 may be activated to obtain blood glucose levels in response to an input user request received by the computing device.

During the monitoring of the blood glucose levels of the patient (step 802), the process 800 detects an abnormality in the sequence of blood glucose values of the patient (step 804). An abnormality in blood glucose values may include blood glucose levels below a predefined hypoglycemia threshold, blood glucose levels above a predefined hyperglycemia threshold, a decreasing blood glucose trend approaching or below a predefined hypoglycemia threshold, an increasing blood glucose trend approaching or above a predefined hyperglycemia threshold, or the like. The abnormality indicates that an intervention is required to maintain blood glucose stability at appropriate levels, and the intervention may include the administration of a bolus of insulin (to decrease hyperglycemic blood glucose levels) or the administration of a dose of carbohydrates (to increase hypoglycemic blood glucose levels).

After detecting the abnormality (step 804), the process 800 determines whether a corrective action has been taken, to address the abnormality (decision 806). In some embodiments, when the corrective action is the administration of a dose of carbohydrates, the corrective action is administered by a user. For example, when a sequence of blood glucose values is decreasing and/or detected below a predefined hypoglycemia threshold, then the patient administers a dose of carbohydrates to change the direction of the blood glucose trend from decreasing to increasing. In this example, the patient may self-administer any form of a dose of carbohydrates, including any type of food, drink, or medication taken orally, and/or any type of blood glucose medication administered intravenously or otherwise. Also in this example, the process 800 determines that a corrective action has been taken by the user by receiving user input data indicating that the corrective action has been taken.

In some embodiments, when the corrective action is the administration of a bolus of insulin, the corrective action is administered by the insulin delivery pump, which is performed in response to a user input command. Again, in this example, the process 800 determines that a corrective action has been taken by the user by receiving user input data indicating that the corrective action has been taken. For example, when a sequence of blood glucose values is increasing and/or detected above a predefined hyperglycemia threshold, then the insulin delivery pump administers a bolus of insulin to change the direction of the blood glucose trend from increasing to decreasing, in response to a user input command to the insulin delivery pump. In this example, the insulin delivery pump and associated computing device may identify the high blood glucose levels (i.e., the abnormality), alert the user to the abnormality, and receive a user input command to administer a bolus of insulin to treat the abnormality. Also in this example, the process 800 determines that a corrective action has been taken by the insulin pump, by receiving user input data indicating that the corrective action has been performed, wherein the insulin pump administered the insulin in response to a user input request.

The corrective action is an intervention, such as the administration of a bolus of insulin or a dose of carbohydrates. Here, the process 800 determines whether an intervention has occurred based on whether user input has been received, wherein the user input communicates that the user has initiated and/or performed the corrective action. In other words, the process 800 determines whether the insulin delivery pump and associated computing device have taken a corrective action to intervene in the detected blood glucose abnormality (via delivering a bolus of insulin in response to a user command), or whether the user has taken a corrective action to intervene in the detected blood glucose abnormality (via self-administration of a dose of carbohydrates, glucose, or other medication).

Under certain circumstances, the process 800 determines that a corrective action has not been taken (the “No” branch of 706). Here, the process 800 has not received user input indicating that the user has initiated the corrective action via the insulin delivery pump and/or that the user has performed the corrective action, and the process 800 therefore determines that the corrective action has not been performed.

When the process 800 determines that a corrective action has not been taken (the “No” branch of 806), then the process 800 presents an alert to prompt user performance of the corrective action, in response to the abnormality (step 808). When the process 800 detects that a corrective action has not been taken, but the corrective action is required due to the detected abnormality (e.g., blood glucose levels that are too high or too low), then the process 800 performs an appropriate step to address the detected abnormality. When the abnormality includes blood glucose levels that are too high, the process 800 prompts the user to administer a bolus of insulin, via the insulin deliver pump, to treat the high blood glucose levels. Here, the process 800 may present an alert to prompt the user to input a command for the insulin delivery pump to administer the bolus of insulin. When the abnormality includes blood glucose levels that are too low, the process 800 prompts the user to administer a dose of carbohydrates to treat the low blood glucose levels.

Under certain circumstances, the process 800 determines that a corrective action has been taken (the “Yes” branch of 806). Here, the process 800 determines that the computing device has received a user input indication that the user has performed a corrective action on his own. When the process 800 determines that a corrective action has been taken (the “Yes” branch of 806), then the process 800 monitors the sequence of blood glucose levels (step 810) to detect any effect resulting from administration of the corrective action on the blood glucose levels. Here, the process 800 continues to obtain, store, and analyze blood glucose levels via the insulin delivery pump and associated computing device.

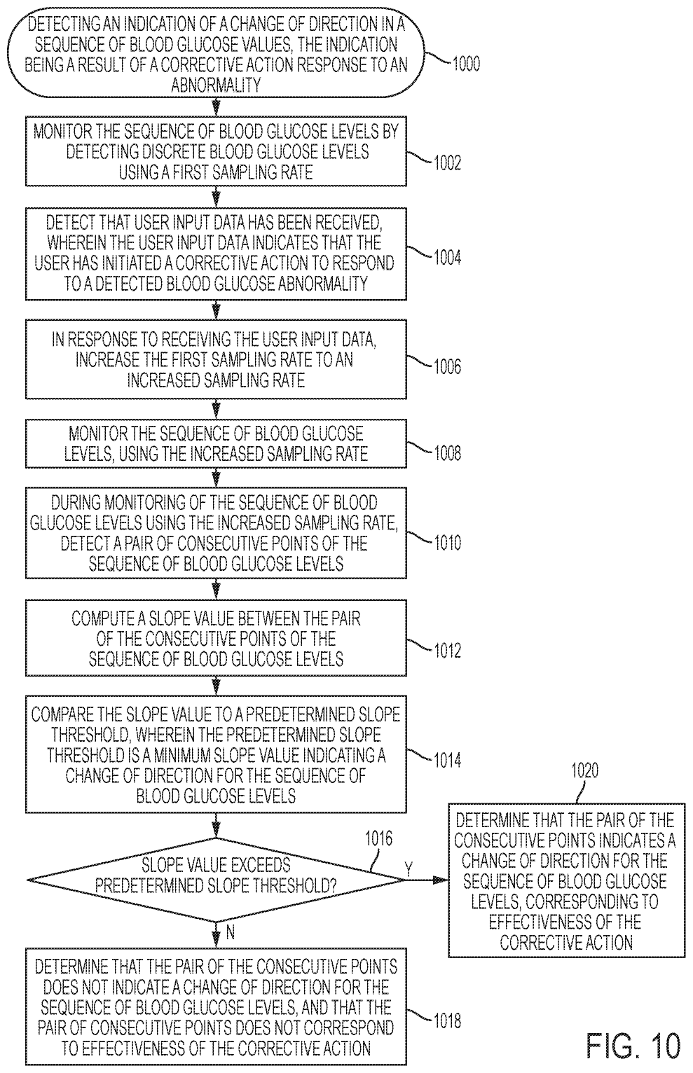

After administration of the corrective action (step 810), the process 800 detects an indication of a change of direction in the blood glucose trend (i.e., the sequence of blood glucose levels) (step 812). One suitable methodology for detecting the indication of the change of direction in the blood glucose trend is described below with reference to FIG. 10. The indication may be referred to as a turn, swerve, curve, inflection arc, and/or any other term designating a change of direction of the blood glucose trend of the patient, wherein the change of direction occurs as a result of the corrective action. The indication of the change of direction in the sequence of blood glucose levels (i.e., the blood glucose trend) includes a plurality of sequential points at a location of the blood glucose trend where a first trend of the blood glucose values alternates direction (at the indication) to create a second trend progressing in an opposite direction of the first trend. In some embodiments, the first trend is increasing or rising, prior to the indication of the change of direction, and progresses in an opposite direction of the second trend, which is decreasing or dropping. In other embodiments, the first trend is decreasing, prior to the indication of the change of direction, and progresses in an opposite direction of the second trend, which is increasing.

In response to the detected indication of a change of direction in blood glucose levels (step 812), the process 800 presents an alert (step 814) via the insulin delivery pump, such that the patient is informed that the administered corrective action has affected the blood glucose levels of the patient in the desired manner. For example, when the corrective action includes the administration of a bolus of insulin to intervene in increasing blood glucose levels approaching and/or above a predefined hyperglycemia threshold, then the indication of the change of direction alerts the user that the insulin has taken effect and caused the blood glucose levels to change direction and begin decreasing. As another example, when the corrective action includes the administration of a dose of carbohydrates to intervene in decreasing blood glucose levels approaching and/or below a predefined hypoglycemia threshold, then the indication of the change of direction of the blood glucose trend alerts the user that the administered carbohydrates have taken effect and caused the blood glucose levels to change direction and begin increasing. Thus, as applied to both examples, the alert provides a notification to the patient that the corrective action has produced the desired effect. The alert may include audio alerts (e.g., alarms or other sound effects), visual alerts (e.g., graphical elements and text presented via user interface or display of the insulin delivery pump), text alerts (e.g., a text-based message transmitted via Short Message Service (SMS), an email message transmitted via the internet), or the like.

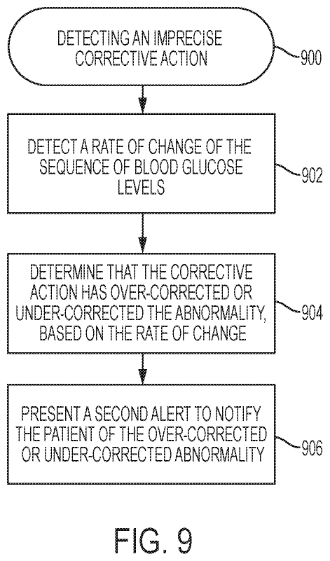

FIG. 9 is a flow chart that illustrates an exemplary embodiment of a process 900 for detecting an imprecise corrective action, in response to a detected blood glucose abnormality, in accordance with the disclosed embodiments. A corrective action is a user-administered or a user-initiated, insulin delivery pump-administered response to treat a blood glucose abnormality (e.g., blood glucose levels that are too high or approaching a hyperglycemia threshold, or blood glucose levels that are too low or approaching a hypoglycemia threshold). Corrective actions may include the administration of a bolus of insulin (to treat high blood glucose levels) or the administration of a dose of carbohydrates, glucose, or other medication (to treat low blood glucose levels).

As described previously with regard to FIG. 8, a corrective action is generally taken to produce an indication of a change of direction for a blood glucose trend, wherein the indication may be referred to as a turn, swerve, curve, inflection arc, or other direction change designation, wherein the change of direction is a shift from a decreasing blood glucose level trend into an increasing blood glucose level trend, or the change of direction is a shift from an increasing blood glucose level trend into a decreasing blood glucose level trend. Thus, the indication alerts the user that the blood glucose abnormality is in the process of being corrected. An imprecise corrective action is a corrective action that does not adequately treat the blood glucose abnormality, such that additional corrective actions are necessary to bring the blood glucose levels of the patient within normal limits. The process 900 may begin at any time after a corrective action has been taken (e.g., following the “Yes” branch of 806, FIG. 8). In this regard, the process 900 may begin during monitoring of the sequence of blood glucose levels (step 810), before or after detection of the indication of the change of direction in the sequence of blood glucose levels (step 812), and before or after presenting an alert based on the detected change of direction (step 814).

First, the process 900 detects a rate of change of the sequence of blood glucose levels (step 902). As described previously, the insulin delivery pump is communicatively coupled to a subcutaneous sensor, and is monitoring blood glucose levels of the user of the insulin delivery pump via the subcutaneous sensor (as described previously, with regard to FIGS. 1-7). As the blood glucose levels are obtained, the process 900 calculates a rate of change for the blood glucose levels.

Next, the process 900 determines that the corrective action has over-corrected or under-corrected the abnormality, based on the rate of change (step 904). In some embodiments, the abnormality is the current existence a hypoglycemic condition, or a condition approaching a hypoglycemic condition, which is indicated by the blood glucose levels of the patient. Here, a monitored sequence of blood glucose levels is below a predefined hypoglycemic threshold or decreasing toward a predefined hypoglycemic threshold, indicating that a corrective action is required. In other embodiments, the abnormality is the current existence a hyperglycemic condition, or a condition approaching a hyperglycemic condition, which is indicated by the blood glucose levels of the patient. Here, a monitored sequence of blood glucose levels is above a predefined hyperglycemic threshold or increasing toward a predefined hyperglycemic threshold, indicating that a corrective action is required.

Over-correction of the abnormality may be the administration of more than a required amount of insulin (which decreases blood glucose values by more than a required amount) or the administration of more than a required dose of carbohydrates (which increases blood glucose values by more than a required amount). Under-correction of the abnormality may be the administration of less than a required amount of insulin (which decreases blood glucose values by less than a required amount) or the administration of less than a required dose of carbohydrates (which increases blood glucose values by less than a required amount). The desired result of any corrective action is a change to the blood glucose levels of the patient such that the blood glucose levels are altered, and after being altered, exist within normal limits. Here, the process 900 determines that, although the corrective action has been administered, the rate of change of the blood glucose values indicate that the corrective action was not drastic enough to produce the desired result of bringing the blood glucose levels into normal limits, or that the corrective action was too drastic and raised or lowered the blood glucose levels beyond normal limits.

The process 900 then presents a second alert to notify the patient of the over-corrected or under-corrected abnormality (step 906). As described herein, the process 900 presents an alert via the insulin delivery pump, such that the patient is informed that the administered corrective action has not affected the blood glucose levels of the patient in the desired manner (e.g., that the corrective action has over-corrected or under-corrected the abnormality). The alert may include audio alerts (e.g., alarms or other sound effects), or visual alerts (e.g., graphical elements and text presented via user interface or display of the insulin delivery pump).

FIG. 10 is a flow chart that illustrates an embodiment of a process 1000 for detecting an indication of a change of direction in a sequence of blood glucose levels, the indication of the change of direction being a result of a corrective action response to an abnormality, in accordance with the disclosed embodiments. It should be appreciated that the process 1000 described in FIG. 10 represents one embodiment of steps 810-812 described above in the discussion of FIG. 8, including additional detail.

The process 1000 monitors a sequence of blood glucose levels by detecting discrete blood glucose levels using a first sampling rate (step 1002). As described herein, the insulin delivery pump is communicatively coupled to a subcutaneous sensor, and is monitoring blood glucose levels of the user of the insulin delivery pump via the subcutaneous sensor (as described previously, with regard to FIGS. 1-7). The first sampling rate used by the insulin delivery pump is a preconfigured value specific to each type of insulin delivery pump. Typically, sampling rates are manufacturer-specific and/or design-specific and are not user-configurable values.

The process 1000 also detects that user input data has been received, wherein the user input data indicates that the user has initiated and/or performed a corrective action to respond to a detected blood glucose abnormality (step 1004). The user input data is described previously with regard to decision 806 of FIG. 8, and is received by the process 1000 via a user interface of a computing device and/or an insulin delivery pump.

In response to the user input data indicating that a corrective action has been initiated and/or performed by the user (step 1004), the process 1000 increases the first sampling rate to an increased sampling rate (step 1006), and the process 1000 monitors the sequence of blood glucose levels, using the increased sampling rate (step 1008). The first sampling rate used by the insulin delivery pump is a sampling rate configured to accommodate battery life and other parameters of the insulin delivery pump and associated computing device. The sampling rate is increased such that the process 1000 is capable of detecting the indication of the change of direction of the blood glucose trend more quickly and with greater detail.

During monitoring of the sequence of blood glucose levels, the process 1000 detects a plurality of sequential points of the sequence of blood glucose levels (step 1010). Each of the plurality of sequential points is a discrete blood glucose value, obtained consecutively over time, via the blood glucose sensor. The process 1000 then computes a slope value between a pair of the sequential points of the blood glucose trend (step 1012). As described herein, slope values describe a steepness of the blood glucose trend between a particular pair of discrete blood glucose values (i.e., two sequential points of the blood glucose trend), and the slope values are calculated using a ratio of vertical change to horizontal change (i.e., “rise over run”). Here, the pair of the sequential points includes a first sequential point and a second sequential point, wherein the first sequential point and the second sequential point are obtained consecutively and in order, via the blood glucose sensor.

The process 1000 then compares the slope value to a predetermined slope threshold (step 1014), wherein the predetermined slope threshold is a minimum slope value indicating a change of direction for the blood glucose trend. When the slope value does not exceed the predetermined slope threshold (the “No” branch of 1016), then the process 1000 determines that the pair of the sequential points does not indicate a change of direction for the blood glucose trend (step 1018), and thus the corrective action has not yet become effective to produce the desired change at the location of the pair of the sequential points in the blood glucose trend.

When the slope value exceeds the predetermined slope threshold (the “Yes” branch of 1016), then the process 1000 determines that the pair of the sequential points indicates a change of direction for the blood glucose trend (step 1020). Here, the slope value is large enough that the process 1000 determines that an increasing blood glucose trend progressing in a first direction has changed direction to progress in an opposite direction and become a decreasing blood glucose trend, or the process 1000 determines that a decreasing blood glucose trend progressing in a first direction has changed direction to progress in an opposite direction and become an increasing blood glucose trend. Thus, the corrective action is determined to be effective to alter the current state of the blood glucose trend (which includes an abnormality, as described herein) at the location of the pair of the sequential points in the blood glucose trend.

The various tasks performed in connection with processes 800-1000 may be performed by software, hardware, firmware, or any combination thereof. For illustrative purposes, the preceding descriptions of processes 800-1000 may refer to elements mentioned above in connection with FIGS. 1-7. In practice, portions of processes 800-1000 may be performed by different elements of the described system. It should be appreciated that processes 800-1000 may include any number of additional or alternative tasks, the tasks shown in FIGS. 8-10 need not be performed in the illustrated order, and processes 800-1000 may be incorporated into a more comprehensive procedure or process having additional functionality not described in detail herein. Moreover, one or more of the tasks shown in FIGS. 8-10 could be omitted from embodiments of the processes 800-1000 as long as the intended overall functionality remains intact.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processor devices can carry out the described operations, tasks, and functions by manipulating electrical signals representing data bits at memory locations in the system memory, as well as other processing of signals. The memory locations where data bits are maintained are physical locations that have particular electrical, magnetic, optical, or organic properties corresponding to the data bits. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. The program or code segments can be stored in a processor-readable medium or transmitted by a computer data signal embodied in a carrier wave over a transmission medium or communication path. The “computer-readable medium”, “processor-readable medium”, or “machine-readable medium” may include any medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like.

The preceding description refers to elements or nodes or features being “connected” or “coupled” together. As used herein, unless expressly stated otherwise, “coupled” means that one element/node/feature is directly or indirectly joined to (or directly or indirectly communicates with) another element/node/feature, and not necessarily mechanically. Likewise, unless expressly stated otherwise, “connected” means that one element/node/feature is directly joined to (or directly communicates with) another element/node/feature, and not necessarily mechanically. Thus, although the schematic shown in FIG. 1 depicts one exemplary arrangement of elements, additional intervening elements, devices, features, or components may be present in an embodiment of the depicted subject matter.

For the sake of brevity, conventional techniques related to signal processing, data transmission, signaling, network control, and other functional aspects of the systems (and the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in an embodiment of the subject matter.

Some of the functional units described in this specification have been referred to as “modules” in order to more particularly emphasize their implementation independence. For example, functionality referred to herein as a module may be implemented wholly, or partially, as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like. Modules may also be implemented in software for execution by various types of processors. An identified module of executable code may, for instance, comprise one or more physical or logical modules of computer instructions that may, for instance, be organized as an object, procedure, or function. Nevertheless, the executables of an identified module need not be physically located together, but may comprise disparate instructions stored in different locations that, when joined logically together, comprise the module and achieve the stated purpose for the module. Indeed, a module of executable code may be a single instruction, or many instructions, and may even be distributed over several different code segments, among different programs, and across several memory devices. Similarly, operational data may be embodied in any suitable form and organized within any suitable type of data structure. The operational data may be collected as a single data set, or may be distributed over different locations including over different storage devices, and may exist, at least partially, merely as electronic signals on a system or network.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method for confirming effectiveness of therapy provided by an insulin infusion system comprising a computing device communicatively coupled to an insulin delivery pump and a blood glucose sensor, the method comprising:
in response to a corrective action for a detected abnormality in a blood glucose level of a patient,
monitoring a sequence of blood glucose levels of the patient, following the corrective action, by the computing device via the blood glucose sensor;
detecting an indication that the corrective action is effective to correct the detected abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and
initiating presentation of an alert in response to detecting the indication, by the computing device via a communicatively coupled display device.

2. The method of claim 1, further comprising:
detecting a hypoglycemic blood glucose level, by the computing device via the blood glucose sensor, wherein the detected abnormality comprises the hypoglycemic blood glucose level;
receiving user input data communicating that the corrective action has occurred, by the computing device via a user interface;
after receiving the user input data, monitoring the sequence of blood glucose levels, by the computing device via the blood glucose sensor; and
detecting the indication of the change of direction in the sequence of blood glucose levels when a decreasing trend of the sequence of blood glucose levels shifts to create an increasing trend of the sequence of blood glucose levels, by the computing device, wherein the first trend comprises a decreasing trend, and wherein the second trend comprises the increasing trend.

3. The method of claim 1, further comprising:
detecting a hyperglycemic blood glucose level, by the computing device via the blood glucose sensor, wherein the detected abnormality comprises the hyperglycemic blood glucose level;
receiving user input data communicating that a user has initiated insulin delivery via the insulin delivery pump to treat the hyperglycemic blood glucose level, by the computing device via a user interface, wherein the corrective action comprises the insulin delivery;
after receiving the user input data, monitoring the sequence of blood glucose levels, by the computing device via the blood glucose sensor; and
detecting the indication of the change of direction in the sequence of blood glucose levels when an increasing trend of the sequence of blood glucose levels shifts to create a decreasing trend of the sequence of blood glucose levels, by the computing device, wherein the first trend comprises the increasing trend, and wherein the second trend comprises a decreasing trend.

4. The method of claim 1, wherein, further in response to the corrective action, the method further comprises:
detecting a rate of change of the sequence of blood glucose levels, by the computing device;
determining that the corrective action has over-corrected the detected abnormality, based on the rate of change, by the computing device, to create an over-corrected abnormality; and
initiating presentation of a second alert to notify the patient of the over-corrected abnormality, by the computing device.

5. The method of claim 1, wherein, further in response to the corrective action, the method further comprises:
detecting a rate of change of the sequence of blood glucose levels, by the computing device;
determining that the corrective action has under-corrected the detected abnormality, based on the rate of change, by the computing device, to create an under-corrected abnormality; and
initiating presentation of a second alert to notify the patient of the under-corrected abnormality, by the computing device.

6. The method of claim 1, wherein detecting the indication further comprises:
monitoring the sequence of blood glucose levels by detecting discrete blood glucose levels using a first sampling rate, by the computing device via the blood glucose sensor;
detecting that user input data has been received, wherein the user input data indicates that a user has initiated the corrective action to respond to the detected abnormality; and
in response to detecting that the user input data has been received,
increasing the first sampling rate to an increased sampling rate, by the computing device via the blood glucose sensor;
continuing to monitor the sequence of blood glucose levels, using the increased sampling rate, by the computing device via the blood glucose sensor; and
detecting the indication of the change of direction using the increased sampling rate, by the computing device.

7. The method of claim 6, further comprising:
during monitoring the sequence of blood glucose levels using the increased sampling rate, detecting a pair of consecutive points of the sequence of blood glucose levels;
computing a slope value between the pair of consecutive points of the sequence of blood glucose levels;
comparing the slope value to a predetermined slope threshold; and
when the slope value exceeds the predetermined slope threshold, determining that the pair of consecutive points corresponds to effectiveness of the corrective action, wherein the indication comprises at least the pair of consecutive points.

8. The method of claim 7, further comprising:
when the slope value does not exceed the predetermined slope threshold, determining that the pair of consecutive points does not correspond to effectiveness of the corrective action, wherein the indication comprises at least the pair of consecutive points.

9. A medical device system comprising:
an insulin delivery system;
a glucose sensor system; and
a computing device comprising a system memory element and at least one processor, the computing device communicatively coupled to the insulin delivery system and the glucose sensor system, and the computing device configured to:
detect an abnormality in a blood glucose level of a patient, via the glucose sensor system; and
in response to a corrective action for the abnormality,
monitor a sequence of blood glucose levels of the patient, following the corrective action, via the glucose sensor system;
detect an indication that the corrective action is effective to correct the abnormality, by the computing device, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and
in response to detecting the indication, present an alert via the insulin delivery system.

10. The medical device system of claim 9, wherein the computing device is further configured to:
detect a hypoglycemic blood glucose level, via the glucose sensor system, wherein the abnormality comprises the hypoglycemic blood glucose level;
receive user input data communicating that the corrective action has occurred, via a user interface of the insulin delivery system;
after receiving the user input data, monitor the sequence of blood glucose levels, via the glucose sensor system; and
detect the indication of the change of direction in the sequence of blood glucose levels when a decreasing trend of the sequence of blood glucose levels shifts to create an increasing trend of the sequence of blood glucose levels, wherein the first trend comprises a decreasing trend, and wherein the second trend comprises the increasing trend.

11. The medical device system of claim 9, wherein the computing device is further configured to:
detect a hyperglycemic blood glucose level, via the glucose sensor system, wherein the abnormality comprises the hyperglycemic blood glucose level;
receive user input data communicating that a user has initiated insulin delivery by the insulin delivery system to treat the hyperglycemic blood glucose level, via a user interface, wherein the corrective action comprises the insulin delivery;
after receiving the user input data, monitor the sequence of blood glucose levels, via the glucose sensor system; and
detect the indication of the change of direction in the sequence of blood glucose levels when an increasing trend of the sequence of blood glucose levels shifts to create a decreasing trend of the sequence of blood glucose levels, wherein the first trend comprises the increasing trend, and wherein the second trend comprises a decreasing trend.

12. The medical device system of claim 9, wherein the computing device is further configured to:
detect a rate of change of the sequence of blood glucose levels;
determine that the corrective action has over-corrected the abnormality, based on the rate of change, to create an over-corrected abnormality; and
initiate presentation of a second alert to notify the patient of the over-corrected abnormality.

13. The medical device system of claim 9, wherein the computing device is further configured to:
detect a rate of change of the sequence of blood glucose levels;
determine that the corrective action has under-corrected the abnormality, based on the rate of change, to create an under-corrected abnormality; and
initiate presentation of a second alert to notify the patient of the under-corrected abnormality.

14. The medical device system of claim 9, wherein the computing device is further configured to:
monitor the sequence of blood glucose levels by detecting discrete blood glucose levels using a first sampling rate, via the glucose sensor system;
detect that user input data has been received, wherein the user input data indicates that a user has initiated the corrective action to respond to the abnormality; and
in response to detecting that the user input data has been received,
increase the first sampling rate to an increased sampling rate, via the glucose sensor system;
continue to monitor the sequence of blood glucose levels, using the increased sampling rate, via the glucose sensor system; and
detect the indication of the change of direction using the increased sampling rate, via the glucose sensor system.

15. The medical device system of claim 14, wherein the computing device is further configured to:
during monitoring the sequence of blood glucose levels using the increased sampling rate, detect a pair of consecutive points of the sequence of blood glucose levels;
compute a slope value between the pair of consecutive points of the sequence of blood glucose levels;
compare the slope value to a predetermined slope threshold; and
when the slope value exceeds the predetermined threshold, determine that the pair of consecutive points corresponds to effectiveness of the corrective action, wherein the indication comprises at least the pair of consecutive points.

16. An insulin delivery system comprising:
a glucose sensor system; and
an insulin infusion device comprising an integrated computing device, the insulin infusion device configured to:
detect an abnormality in a blood glucose level of a patient, via the glucose sensor system; and
in response to a corrective action for the abnormality,
monitor a sequence of blood glucose levels of the patient, following the corrective action, via the glucose sensor system;
detect an indication that the corrective action is effective to correct the abnormality, wherein the indication comprises a change of direction in the sequence of blood glucose levels, wherein a first trend in the sequence of blood glucose levels shifts at the indication to create a second trend in blood glucose levels, the second trend progressing in an opposite direction of the first trend; and
in response to detecting the indication, present an alert.

17. The insulin delivery system of claim 16, wherein the insulin infusion device is further configured to:
detect a hypoglycemic blood glucose level, via the glucose sensor system, wherein the abnormality comprises the hypoglycemic blood glucose level;
receive user input data communicating that the corrective action has occurred, via a user interface of the insulin infusion device;
after receiving the user input data, monitor the sequence of blood glucose levels, via the glucose sensor system; and
detect the indication of the change of direction in the sequence of blood glucose levels when a decreasing trend of the sequence of blood glucose levels shifts to create an increasing trend of the sequence of blood glucose levels, wherein the first trend comprises the decreasing trend, and wherein the second trend comprises the increasing trend.

18. The insulin delivery system of claim 16, wherein the insulin infusion device is further configured to:
detect a hyperglycemic blood glucose level, via the glucose sensor system, wherein the abnormality comprises the hyperglycemic blood glucose level;
receive user input data communicating that a user has initiated insulin delivery by the insulin delivery system to treat the hyperglycemic blood glucose level, via a user interface, wherein the corrective action comprises the insulin delivery;
after receiving the user input data, monitor the sequence of blood glucose levels, via the glucose sensor system; and
detect the indication of the change of direction in the sequence of blood glucose levels when an increasing trend of the sequence of blood glucose levels shifts to create a decreasing trend of the sequence of blood glucose levels, wherein the first trend comprises the increasing trend, and wherein the second trend comprises the decreasing trend.

19. The insulin delivery system of claim 16, wherein the insulin infusion device is further configured to:
monitor the sequence of blood glucose levels by detecting discrete blood glucose levels using a first sampling rate, via the glucose sensor system;
detect that user input data has been received, wherein the user input data indicates that a user has initiated the corrective action to respond to the abnormality; and
in response to detecting that the user input data has been received,
increase the first sampling rate to an increased sampling rate, via the glucose sensor system;
continue to monitor the sequence of blood glucose levels, using the increased sampling rate, via the glucose sensor system; and
detect the indication of the change of direction using the increased sampling rate, via the glucose sensor system.

20. The insulin delivery system of claim 19, wherein the insulin infusion device is further configured to:
during monitoring the sequence of blood glucose levels using the increased sampling rate, detect a pair of consecutive points of the sequence of blood glucose levels;
compute a slope value between the pair of consecutive points of the sequence of blood glucose levels;
compare the slope value to a predetermined slope threshold; and
when the slope value exceeds the predetermined threshold, determine that the pair of consecutive points corresponds to effectiveness of the corrective action, wherein the indication comprises at least the pair of consecutive points.
